(19) United States
(12) Patent Application Publication
(10) Pub. No.: US 2013/0316067 A1
(43) Pub. Date: Nov. 28, 2013

(54) METHOD FOR PRODUCING GAMMA-AMINOBUTYRIC ACID BY FERMENTING RICE MEDIUM FROM PREPARATION OF CORDYCEPS SPECIES AND APPLICATION THEREOF

(71) Applicant: NATIONAL TAIWAN OCEAN UNIVERSITY, Keelung City (TW)

(72) Inventors: GUO-JANE TSAI, Taipei City (TW); I-CHIN HUNG, Tainan City (TW)

(73) Assignee: NATIONAL TAIWAN OCEAN UNIVERSITY, Keelung City (TW)

(21) Appl. No.: 13/899,692

(22) Filed: May 22, 2013

(30) Foreign Application Priority Data
May 23, 2012 (TW) 101118312

(51) Int. Cl. C12P 13/00; A23L 1/28

(52) U.S. Cl. CPC C12P 13/005; A23L 1/28
USPC 426/590; 435/128; 426/656

(57) ABSTRACT

The present invention provides a method for producing γ-aminobutyric acid by fermenting a rice medium from the preparation of a Cordyceps species and the application thereof. Firstly, water and an appropriate amount of rice medium are mixed together to form a fermentation broth. Secondly, add lactic acid bacteria to the fermentation broth. Then, the fermentation broth is fermented by both of the Cordyceps species retained in the rice medium and the lactic acid bacteria. Consequently, the γ-aminobutyric acid is produced. Moreover, the fermentation broth prepared by this method can be applied to a health food or drink.

### FIELD OF THE INVENTION

The present invention relates to a method for producing γ-aminobutyric acid, and more particularly to a method for producing highly-concentrated γ-aminobutyric acid by fermenting a rice medium which is obtained from the preparation of a Cordyceps species. The present invention also relates to the application of the γ-aminobutyric acid.

### BACKGROUND OF THE INVENTION

Taxonomically, Cordyceps is a genus of ascomycete fungi that includes many species. All Cordyceps species are insect-parasitic fungi. The fruiting body and mycelium extract of the Cordyceps species is one of the widely eaten Chinese herbal medicines and health food by people in the East Asia region. For example, some ancient books of traditional Chinese medicine record that the fruiting body and mycelium extract of the Cordyceps are effective in treating the deficiency of Lung Yin and the deficiency of kidney Yang (Lung and kidney are functionally defined entity in Chinese medicine, and are not equivalent to the anatomical organ of the same name. In the classification of Chinese medicine, Lung is a yin organ, and there are two kidneys, kidney yin and kidney yang.), and thus helpful for relieving cough, reducing phlegm, and reducing the insomnia problem caused by deficiency of kidney Yang. The most well-known and widely-used Cordyceps species are Cordyceps sinensis and Cordyceps militaris.

A conventional method for wildly breeding the fruiting bodies of the Cordyceps species will be illustrated as follows. In summer, insects lay eggs, and larvae hatch from these eggs. The larvae will dig into wet and soft soil. Consequently, the larvae are infected by spores of the Cordyceps species within the soil. In the spring of next year, the spores of the Cordyceps species germinate and mycelia grow. The mycelia absorb the substances in the insect body as the nutrient for their survival. Accompanying with the growth of the fungal mycelia, the tissue of the insect body is gradually infiltrated and replaced by the fungal mycelia until the insect dies. The fungal mycelia continuously grow to erupt from the insect body, and finally form the fruiting body in the summer.

However, due to the geographical particularity for Cordyceps cultivation, the stringent parasitic characteristic of Cordyceps, and the over-harvesting of the Cordyceps fungus, the resources of the fruiting bodies of the wild Cordyceps fungus are extremely insufficient.

Recently, the methods for artificially breeding the fruiting bodies of the Cordyceps species become more popular. In the artificial breeding methods, human use a cooked starchy material such as a cooked cereal material (e.g. rice, corn or millet) or a cooked starch-rich plant tissue (e.g. Chinese yam, sweet potato or potato) instead of the insect bodies to cultivate Cordyceps fungus. Cooked cereal material provides nutrient substances to the mycelia of Cordyceps militaris, and also plays a similar role as insect to be erupted by the fruiting bodies of Cordyceps militaris.

FIG. 1 schematically illustrates the outward appearance of fruiting bodies of Cordyceps militaris cultivated with cooked rice. In FIG. 1, a rice medium 1 and fruiting bodies 2 of Cordyceps militaris are shown. The rice medium 1 is completely or partially infiltrated and replaced by the mycelia of Cordyceps militaris. The fruiting bodies 2 of Cordyceps militaris are cultivated with the rice medium 1 and finally erupting from said rice medium 1. Researches found that the fruiting bodies of Cordyceps militaris artificially cultivated with the cooked rice have similar amount of physiological active substances (e.g. cordycepin, cordycepic acid, ergosterol, Cordyceps polysaccharide or superoxide dismutase) as the fruiting bodies of wildly-cultivated Cordyceps militaris. Consequently, using cooked rice as a cultivating medium has become a popular method for artificially breeding the fruiting bodies of Cordyceps militaris.

However, this artificially breeding method still has some drawbacks since it produces lots of waste and the nutrients remaining cannot be used. FIG. 2 is a residual rice medium, the remaining rice medium after the fruiting bodies as shown in FIG. 1 are cut off. After the fruiting bodies are cut off to be sold, a large amount of residual rice medium infiltrated by the fungal mycelia will be remained. If the residual rice medium is discarded directly, it is detrimental to the ecological environment and may pollute the soil. On the other hand, the residual rice medium still contains many healthily-valuable mycelia of the Cordyceps species. So, the residual rice medium may be a good food resource. It is a pity to burn the residual rice medium to ashes and then discard the ashes.

No one eats the residual rice medium as shown in FIG. 2 probably because its nutritional value and health value are much inferior to the fruiting bodies of the Cordyceps species. Moreover, the residual rice medium has a sour taste and unpleasant smell, so it is hard to become a food. Therefore, it is an important and commercially-valuable subject to effectively deal with the residual rice medium, reuse the nutrient substances and the Cordyceps fungal mycelia in the residual rice medium, and obviously increase the health value and edibility thereof while making good use of the residual rice medium from the cultivation of the Cordyceps fruiting bodies.

As known, the Cordyceps species may be used as health food for nourishing lung Yin and reinforcing kidney Yang in order for relieving cough, reducing phlegm, and reducing the insomnia problem caused by deficiency of kidney Yang. Through wholehearted experience and research, the inventors of the present invention provide a fermentation method to convert the component in the residual rice medium into a much higher value component-γ-aminobutyric acid (GABA) by using edible probiotic. Since γ-aminobutyric acid is effective in reducing the insomnia problem and in helping relieve symptoms of depression just similar as Cordyceps, the residual rice medium may become a much higher health value food or drinking through this fermentation method. In other words, the present invention provides a novel method for producing γ-aminobutyric acid and the use of γ-aminobutyric acid in food.

### SUMMARY OF THE INVENTION

The present invention provides a method for producing γ-aminobutyric acid by fermenting a rice medium.

The present invention also provides a health food or drink containing γ-aminobutyric acid that is produced by fermenting a rice medium.

In accordance with an aspect of the present invention, the present invention provided a method for producing γ-aminobutyric acid. The method includes the following steps; and

Firstly, preparing a suspension of a residual rice medium. Then, fermenting said suspension with a living Cordyceps species retained in said residual rice medium and/or with an externally-added microorganism to produce said γ-aminobutyric acid.

In an embodiment, the residual rice medium is further ground into rice medium powders, and the rice medium powders are mixed with a liquid to form the suspension.

In an embodiment, the residual rice medium is partially or completely infiltrated and replaced by mycelia of the Cordyceps species.

In an embodiment, a crude protein content of the residual rice medium is higher than a crude protein content of the same type of original cooked rice medium material where no fruiting bodies of the Cordyceps species have been cultivated.

In an embodiment, the crude protein content of the residual rice medium is 6%˜30% on a dry weight basis.

In an embodiment, a weight/volume percent concentration of the residual rice medium of the suspension is lower than or equal to 60%.

In an embodiment, the externally-added microorganism is a lactic acid bacteria.

In an embodiment, the lactic acid bacteria is Lactobacillus brevis.

In an embodiment, the externally-added microorganism is an externally-added Cordyceps species.

In an embodiment, a weight/volume percent concentration of the externally-added Cordyceps species of the suspension is lower than or equal to 50%.

In accordance with another aspect of the present invention, there is provided a food or drink. The food or drink contains γ-aminobutyric acid and cordycepin.

In an embodiment, the concentration of the γ-aminobutyric acid is higher than 0.1 mg/mL. Alternatively, the concentration of the γ-aminobutyric acid is higher than 0.1 mg/mL and the concentration of the cordycepin is higher than 10 μg/mL, or the concentration of said γ-aminobutyric acid is higher than 0.3 mg/g and the concentration of said cordycepin is higher than 30 μg/g.

In an embodiment, the concentration of the γ-aminobutyric acid is higher than 3 mg/mL, or the concentration of said γ-aminobutyric acid is higher than 9 mg/g.

In an embodiment, the concentration of the cordycepin is higher than 30 μg/mL, or the concentration of said cordycepin is higher than 90 μg/g.

In an embodiment, at least partial of said γ-aminobutyric acid is the product from fermentating a residual rice medium.

In an embodiment, further comprising an edible material for harmonizing the odors or tastes of said residual rice medium, and/or for harmonizing the the odors or tastes from fermentation.

In an embodiment, said edible material is at least one selected from Chinese red date, dried longan, and edible Chinese herbal medicine.

In an embodiment, said edible material is at least one selected from a Chinese red date extract, a dried longan extract, and an edible Chinese herbal medicine extract.

In accordance with a further aspect of the present invention, there is provided a material for preparing a food or drink. The material includes a starchy material and fungal mycelia and/or cordycepin. The starchy material is obtained from a plant tissue and/or from a cereal.

In an embodiment, the concentration of said cordycepin is higher than 90 μg/g, or the concentration of said cordycepin is higher than 30 μg/mL

In an embodiment, the starchy material is a residual starchy medium. The residual starchy medium is obtained by growing a Cordyceps species on a starchy medium and cutting off fruiting bodies of the Cordyceps species from the starchy medium to leave the residual starchy medium.

The above objects and advantages of the present invention will become more readily apparent to those ordinarily skilled in the art after reviewing the following detailed description and accompanying drawings, in which:

### BRIEF DESCRIPTION OF THE DRAWINGS

FIG. 1 schematically illustrates the outward appearance of fruiting bodies of Cordyceps militaris cultivated with cooked rice;

FIG. 2 is a residual rice medium, the remaining rice medium after the fruiting bodies as shown in FIG. 1 are cut off;

FIG. 3 is a flowchart illustrating a method for producing γ-aminobutyric acid by fermenting a rice medium according to a first embodiment of the present invention;

FIG. 4 is a flowchart illustrating a method for producing γ-aminobutyric acid by fermenting a rice medium according to a second embodiment of the present invention;

FIG. 5 is the thin layer chromatography result showing the γ-aminobutyric acid produced by ten lactic acid bacteria strains;

FIG. 6A is a bar chart showing the total counts of each lactic acid bacteria strain after it ferments the suspension containing 12% of rice medium;

FIG. 6B is a bar chart showing the content of γ-aminobutyric acid after each lactic acid bacteria strain ferments the suspension containing 12% of rice medium;

FIG. 7A is a bar chart showing the total counts of the FPA 3709 strain after it ferments the suspension containing different concentration of rice medium;

FIG. 7B is a bar chart showing the content of γ-aminobutyric acid after the FPA 3709 strain ferments the suspension containing different concentration of rice medium;

FIG. 8 is a bar chart showing the content of γ-aminobutyric acid after the FPA 3709 strain ferments the rice medium suspensions containing different concentrations of dried fruiting bodies powder;

FIG. 9 is a bar chart showing the content of γ-aminobutyric acid after the FPA 3709 strain ferments the rice medium suspensions containing different concentrations of fresh fruiting bodies;

FIG. 10A is a bar chart showing the total counts of the FPA 3709 strain after it ferments the rice medium suspensions containing different concentrations of dried longan or Chinese red date; and

FIG. 10B is a bar chart showing the content of γ-aminobutyric acid after the FPA 3709 strain ferments the rice medium suspensions containing different concentrations of dried longan or Chinese red date.

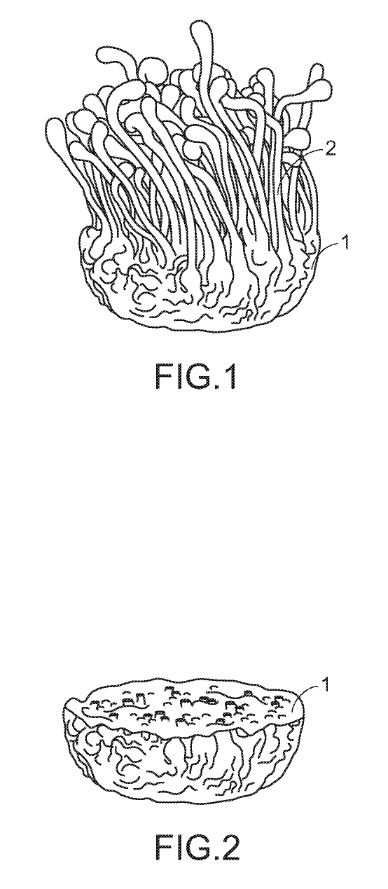

### DETAILED DESCRIPTION OF THE PREFERRED EMBODIMENT

For eliminating the drawbacks from the residual rice medium in the prior art, the present invention provides a method for producing γ-aminobutyric acid by fermenting the residual rice medium with probiotics and the applications thereof. Consequently, a large amount of the residual rice medium can be effectively processed without wasting resources.

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

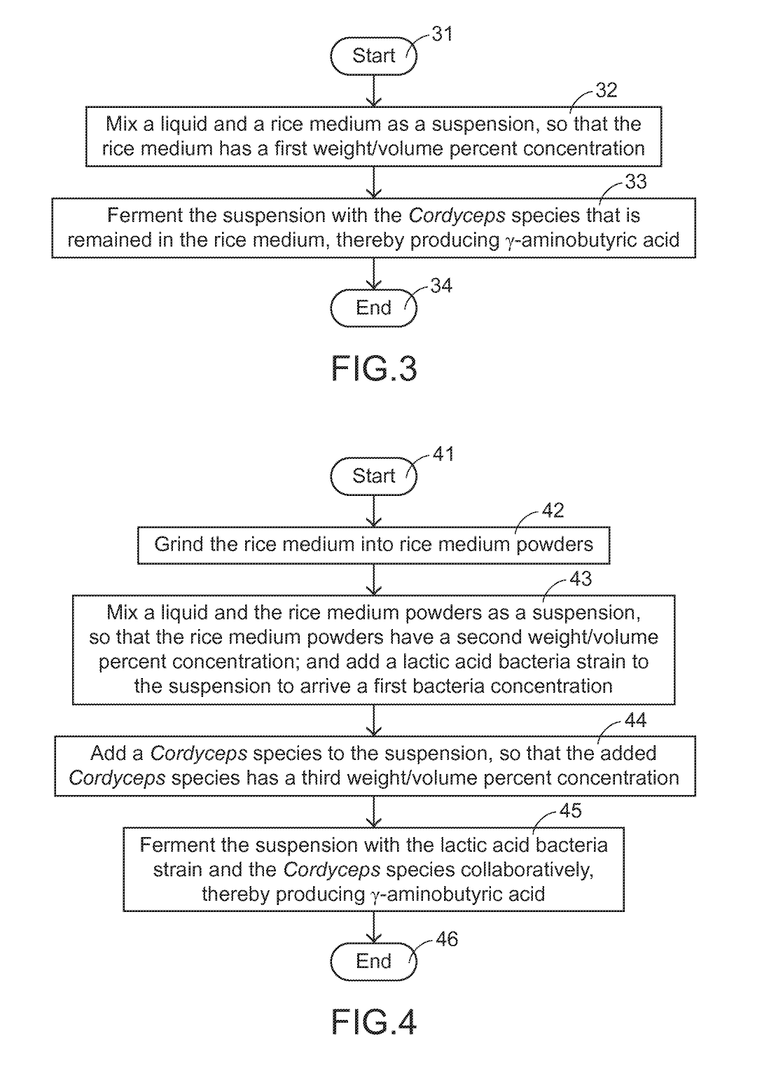

FIG. 3 is a flowchart illustrating a method for producing γ-aminobutyric acid by fermenting a rice medium according to a first embodiment of the present invention. The method at least comprises the following steps. After the flowchart is started (Step 31), a liquid and a rice medium are mixed as a suspension, so that the rice medium has a first weight/volume percent concentration (Step 32). Then, γ-aminobutyric acid is produced by fermenting the suspension with the Cordyceps species that is remained in the rice medium (Step 33). Then, the flowchart is ended (Step 34).

In the step 32 of the method of this embodiment, the liquid and the rice medium containing the Cordyceps species are mixed as the rice medium suspension. The rice medium suspension is responsible for providing the nutrient for the subsequent fermentation process. An example of the liquid includes but is not limited to pure water or any other liquid solution. Preferably, the first weight/volume percent concentration (i.e. the grams of the rice medium/the milliliter of liquid) is lower than or equal to 60%.

In the step 33 of the method of this embodiment, γ-aminobutyric acid is produced by fermenting the suspension with the Cordyceps species that are remained in the rice medium. Preferably, the fermentation process is carried out at 15˜37° for 8˜120 hours.

FIG. 4 is a flowchart illustrating a method for producing γ-aminobutyric acid by fermenting a rice medium according to a second embodiment of the present invention. The method at least comprises the following steps. After the flowchart is started (Step 41), the rice medium is ground into rice medium powders (Step 42). Then, in Step 43, a liquid and the rice medium powders are mixed as a rice medium suspension, so that the rice medium powders have a second weight/volume percent concentration. Moreover, a lactic acid bacteria strain is added to the suspension to arrive a first bacterial concentration. Then, a Cordyceps species is added to the suspension, so that the added Cordyceps species has a third weight/volume percent concentration (Step 44). Then, the γ-aminobutyric acid is produced by fermenting the suspension with the lactic acid bacteria strain and the Cordyceps species collaboratively (Step 45). Then, the flowchart is ended (Step 46).

In the step 42 of the method of this embodiment, since the rice medium is ground into the rice medium powders, the rice medium powders are helpful to accelerate the fermentation process.

In the step 43 of the method of this embodiment, the liquid and the rice medium powders containing the Cordyceps species are mixed as the rice medium suspension. The rice medium suspension is responsible for providing the nutrient for the subsequent fermentation process. Moreover, a lactic acid bacteria strain is added to play a role in the fermentation process. The lactic acid bacteria could be any of the following strains, but should have the glutamate decarboxylase (GAD) activity: Lactobacillus species, Leuconostoc species, Streptococcus species, Pediococcus species, Sporolactobacillus species, Enterococcus species, Lactococcus species, Camobacterium species, Vagococcus species, Tetragenococcus species, Bifidobacterium species, Atopobium species, Weissella species, Abiotrophia species, Granulicatella species, Oenococcus species, or Paralactobacillus species. An example of the liquid includes but is not limited to pure water or any other liquid solution. Preferably, the second weight/volume percent concentration (i.e. the grams of the rice medium/the milliliter of liquid) is lower than or equal to 60%. Preferably, the first bacteria concentration is 5.0˜6.0 log CFU/mL.

In the step 44 of the method of this embodiment, the dried fruiting bodies powder of the Cordyceps species or the fresh fruiting bodies of the Cordyceps species are added as another microorganism for the subsequent fermentation process. The third weight/volume percent concentration (i.e. the grams of the added fruiting bodies/the milliliter of the rice medium suspension) is lower than or equal to 50%.

In the step 45 of the method of this embodiment, the γ-aminobutyric acid is produced by fermenting the rice medium suspension with the lactic acid bacteria strain and the Cordyceps species collaboratively. Preferably, the fermentation process is performed at 15˜37° for 8˜120 hours.

For illustrating the detailed evaluation and the testing results of the above embodiments, the present invention will now be described more specifically with reference to the following experiments.

### Experiment 1: Component Analysis of the Rice Medium

In accordance with the Association of Official Analytical Chemists (A.O.A.C.) Official Methods of Analysis (1980), the contents of moisture, ash, crude protein, crude fat, and crude fiber are determined (do experiments in triplicate).

Please refer to the following table 1, which shows the component analysis of a rice medium after fruiting bodies of Cordyceps militaris are cultivated on cooked rice. The analysis results show that the rice medium is mainly composed of carbohydrate (83.59±0.36%) and followed by crude protein (9.24±0.1%), crude fiber (5.61±0.36%), crude fat (1.31±0.03%) and ash (0.25±0.01%). The crude protein content of the rice medium (9.24%, on a dry weight basis) is obviously higher than the general rice protein content (about 7%, on a dry weight basis), which is collected and published by Department of Health, Executive Yuan, R.O.C. (Taiwan). It means that after the cooked rice is infiltrated by the mycelia of the Cordyceps militaris, the residual rice medium has higher protein content. In other words, the protein content of the residual rice medium is obviously higher than the protein content of the original cooked rice medium material where no fruiting bodies of said Cordyceps species have been cultivated. Moreover, as the mycelia of the Cordyceps militaris infiltrate the rice medium to a higher extent, the crude protein content of the rice medium is closer to the crude protein content of the Cordyceps militaris (36.36%, on a dry weight basis).

The above experimental results show that the rice medium infiltrated by a large amount of Cordyceps militaris is protein-rich. Consequently, it may be possible to use the rice medium infiltrated by the Cordyceps militaris to produce γ-aminobutyric acid by fermentation with probiotics (e.g. lactic acid bacteria).

However, since the rice medium contains a lot of fungal mycelia, it may also contain high concentration of metabolic product obtained from the metabolism of the Cordyceps species (such as metabolic waste or cordycepin, a kind of metabolic product that only Cordyceps species could produce). Due to the metabolic product, the physiology activity, growth activity, or other physiology variables of the lactic acid bacteria may be influenced by the fungal mycelia and the metabolic waste. Moreover, competition or inhibition between the fungal mycelia and the lactic acid bacteria may occur; or the fungal mycelia may directly inhibit the producing process of the γ-aminobutyric acid; or the fungal mycelia may decompose γ-aminobutyric acid. Consequently, it is necessary to evaluate if it is possible to produce a large amount of γ-aminobutyric acid through fermenting the rice medium with lactic acid bacteria.

TABLE 1
Component analysis of a rice medium

| Component | Content (% dry weight) |
|---|---|
| Crude protein | 9.24 ± 0.10 |
| Crude fiber | 5.61 ± 0.36 |
| Crude fat | 1.31 ± 0.03 |
| Ash | 0.25 ± 0.01 |
| Carbohydrate | 83.59 ± 0.56 |

### Experiment 2: Screening of Lactic Acid Bacteria Strains

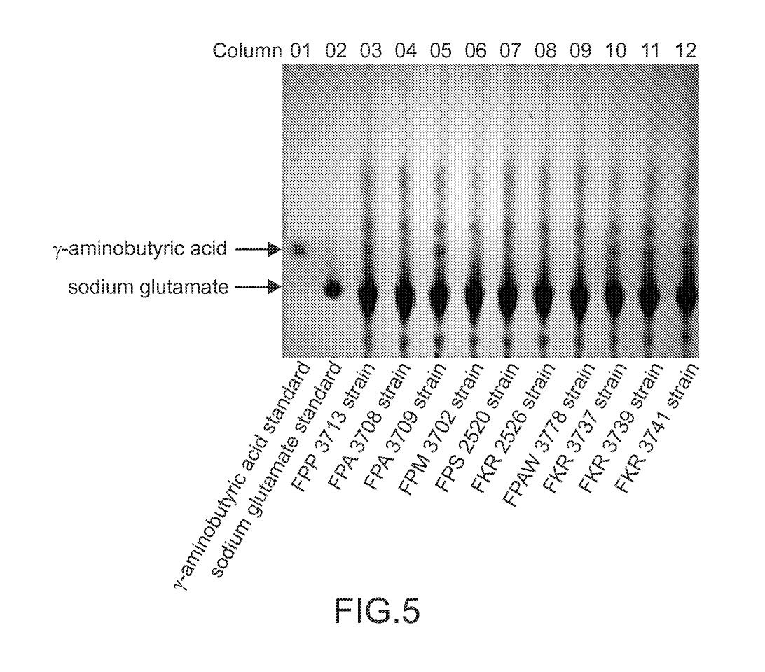

Ten lactic acid bacteria strains having the potentials to produce γ-aminobutyric acid are screened from 126 isolated strains of lactic acid bacteria. After further screening, five strains with the highest productivity of γ-aminobutyric acid are selected. FIG. 5 is the thin layer chromatography result showing the γ-aminobutyric acid produced by ten lactic acid bacteria strains. From left to right of the chromatogram, a γ-aminobutyric acid standard, a sodium glutamate standard, a FPP 3713 strain, a FPA 3708 strain, FPA 3709 strain, FPM 3702 strain, FPS 2520 strain, FKR 2526 strain, FPAW 3778 strain, FKR 3737 strain, FKR 3739 strain, and a FKR 3741 strain are sequentially shown. The experimental results show that the FPA 3709 strain, the FPP 3713 strain, the FKR 3737 strain, the FKR 3739 strain, and the FKR 3741 strain produce more γ-aminobutyric acid. Consequently, these five strains are selected for performing the subsequent experiments.

Experiment 3: Test the γ-aminobutyric Acid Productivity of the Lactic Acid Bacteria in the Rice Medium Suspension

Use a tweezer to put a lump of rice medium into a sterile storage bag, and crush said rice medium into rice medium powders in a mortar (a bowl for grinding materials), which has been subjected to sterilization at 121° C. for 15 minutes. Seal this sterile storage bag with the rice medium powders in it and then stored it in the refrigerator at 4° C. These rice medium powders would be used in preparing the rice medium suspension (in full aseptic treatment).

Then, 12 g of rice medium powders is weighed, and is placed into 100 mL of distilled water, which has been subjected to sterilization at 121° C. for 15 minutes. This is the suspension containing 12% of rice medium. Then, a lactic acid bacteria strain is added to the suspension such that the initial count of the lactic acid bacteria strain is 5.0˜6.0 log CFU/mL. Then, the mixture of the lactic acid bacteria strain and the suspension (also referred as a fermentation broth) is incubated at 37° C. for 48 hours. The total count of the lactic acid bacteria in the fermentation broth is analyzed by a plate count method. The content of the γ-aminobutyric acid in the fermentation broth is analyzed by a high performance liquid chromatography (HPLC) technique.

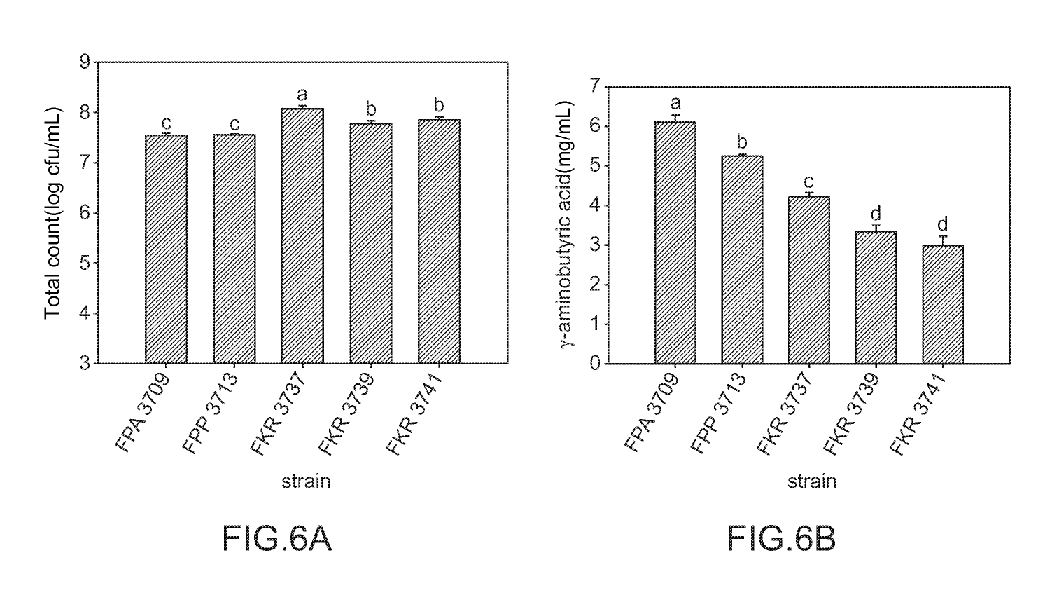

FIG. 6A is a bar chart showing the total counts of each lactic acid bacteria strain after it ferments the suspension containing 12% of rice medium. The horizontal axis as shown in FIG. 6A denotes the FPA 3709 strain, the FPP 3713 strain, the FKR 3737 strain, the FKR 3739 strain, and the FKR 3741 strain sequentially. The vertical axis as shown in FIG. 6A denotes the total count of the lactic acid bacteria. FIG. 6B is a bar chart showing the content of γ-aminobutyric acid after each lactic acid bacteria strain ferments the suspension containing 12% of rice medium. The horizontal axis as shown in FIG. 6B is identical to the horizontal axis as shown in FIG. 6A. The vertical axis as shown in FIG. 6B denotes the content of γ-aminobutyric acid. In FIGS. 6A and 6B, if two groups of data are marked with the same superscript, there is no significant difference between these two groups.

In accordance with the experimental results of FIG. 6A, after the rice medium suspension is fermented by the five lactic acid bacteria strains for 48 hours indepenently, the total count of the FKR 3737 strain is 8.08±0.06 log CFU/mL, which is obviously higher than the total counts of the other four isolated strains. The FPA 3709 strain and the FPP 3713 strain have the fewest total counts (e.g. 7.55±0.04 log CFU/mL and 7.55±0.01 log CFU/mL, respectively). In accordance with the experimental results of FIG. 6B, after the rice medium suspension is fermented by the five lactic acid bacteria strains independently, the productivity of γ-aminobutyric acid by the FPA 3709 strain is the highest (e.g. 6.12±0.18 mg/mL). The productivity of γ-aminobutyric acid by the FKR 3739 strain and the FKR 3741 strain are the lowest (e.g. 3.33±0.16 mg/mL and 2.99±0.24 mg/mL). By comparing the productivity of γ-aminobutyric acid by the above five lactic acid bacteria strains, it is found that the productivity of γ-aminobutyric acid by the FKR 3739 strain is the highest and the productivity of γ-aminobutyric acid by the FPP 3713 is the second highest. Hereinafter, the FKR 3739 isolated strain (e.g. Lactobacillus brevis) is selected to perform the following experiment. It is noted that since FPP 3713 and other three strains could produce γ-aminobutyric acid, they would have the similar results as FKR3739 through the following experiments.

Experiment 4: Effect of the Rice Medium on the γ-aminobutyric Acid Productivity

Firstly, an appropriate amount of rice medium powders is weighed, and is placed into distilled water, which has been subjected to sterilization at 121° C. for 15 minutes. Consequently, 5%, 10%, 15%, 20%, 25%, and 30% (i.e. the grams of the rice medium/the milliliter of water) of rice medium suspensions are prepared. Then, a lactic acid bacteria strain FPA 3709 is added to each rice medium suspension to arrive an initial concentration 5.0˜6.0 log CFU/mL. Then, incubate at 37° C. for 48 hours. The total count of the lactic acid bacteria in the fermentation broth is analyzed by a plate count method. The content of the γ-aminobutyric acid in the fermentation broth is analyzed by a high performance liquid chromatography (HPLC) technique.

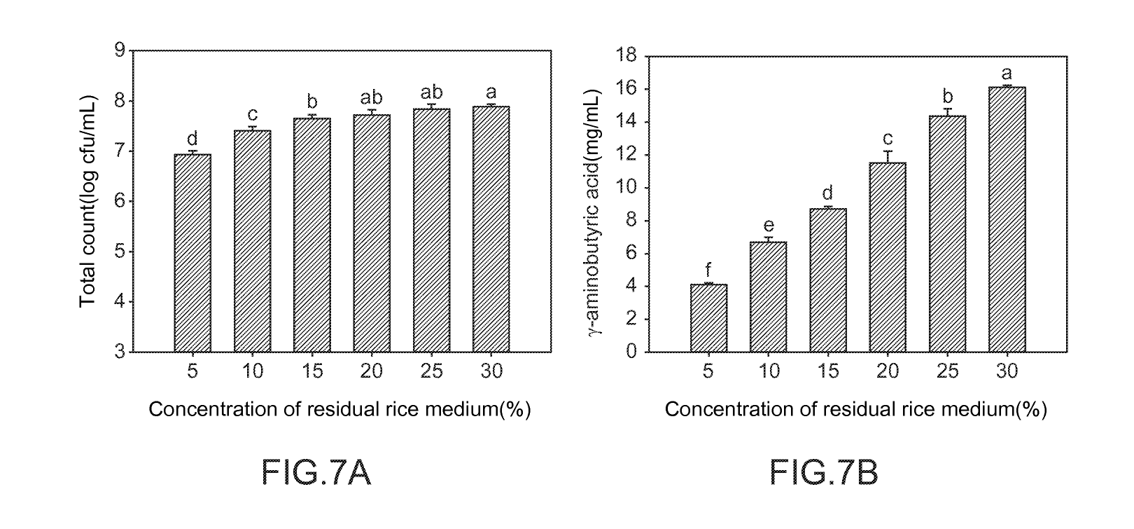

FIG. 7A is a bar chart showing the total counts of the FPA 3709 strain after it ferments the suspension containing different concentration of rice medium. The horizontal axis as shown in FIG. 7A denotes the concentration of the residual rice medium (%, i.e. the grams of the rice medium/the milliliter of water). The vertical axis as shown in FIG. 7A denotes the total count of the lactic acid bacteria strain FPA 3709. FIG. 7B is a bar chart showing the content of γ-aminobutyric acid after the FPA 3709 strain ferments the suspension containing different concentration of rice medium. The horizontal axis as shown in FIG. 7B is identical to the horizontal axis as shown in FIG. 7A. The vertical axis as shown in FIG. 7B denotes the content of γ-aminobutyric acid. In FIGS. 7A and 7B, if two groups of data are marked with the same superscript, there is no significant difference between these two groups.

In accordance with the experimental results of FIG. 7A, as the concentration of the rice medium in the suspension is increased, the total count of the lactic acid bacteria strain FPA 3709 after fermentation is increased. In the group containing 20% of rice medium, the total count of the lactic acid bacteria FPA 3709 is the most (e.g. 7.72±0.10 log CFU/mL). As the concentration of the rice medium in the suspension is continuously increased to 25% or 30%, the total count is nearly kept unchanged. In accordance with the experimental results of FIG. 7B, as the concentration of the rice medium in the suspension is increased, the productivity of γ-aminobutyric acid is increased. In the group containing 30% of rice medium, the productivity of γ-aminobutyric acid is the highest (e.g. 16.12±0.12 mg/mL).

Experiment 5: Effect of the Dried Fruiting Bodies of the Cordyceps Species on the Productivity of γ-aminobutyric Acid

Firstly, an appropriate amount of rice medium powders is weighed, and is placed into distilled water, which has been subjected to sterilization at 121° C. for 15 minutes. Consequently, a 20% of rice medium suspension is prepared. Then, appropriate amounts of dried Cordyceps militaris fruiting bodies powders are added to the rice medium suspension in different bottles. Consequently, the concentrations of the dry fruiting bodies powder in each bottle is 0.4%, 0.8%, 1.2%, 1.6%, and 2.0% (i.e. the grams of the dried fruiting bodies powder/the milliliter of the rice medium suspension). Then, lactic acid bacteria FPA 3709 is added to each rice medium suspension to arrive an initial concentration 5.0˜6.0 log CFU/mL. Then, incubate at 37° C. for 48 hours. The content of the γ-aminobutyric acid in the fermentation broth is analyzed by a high performance liquid chromatography (HPLC) technique.

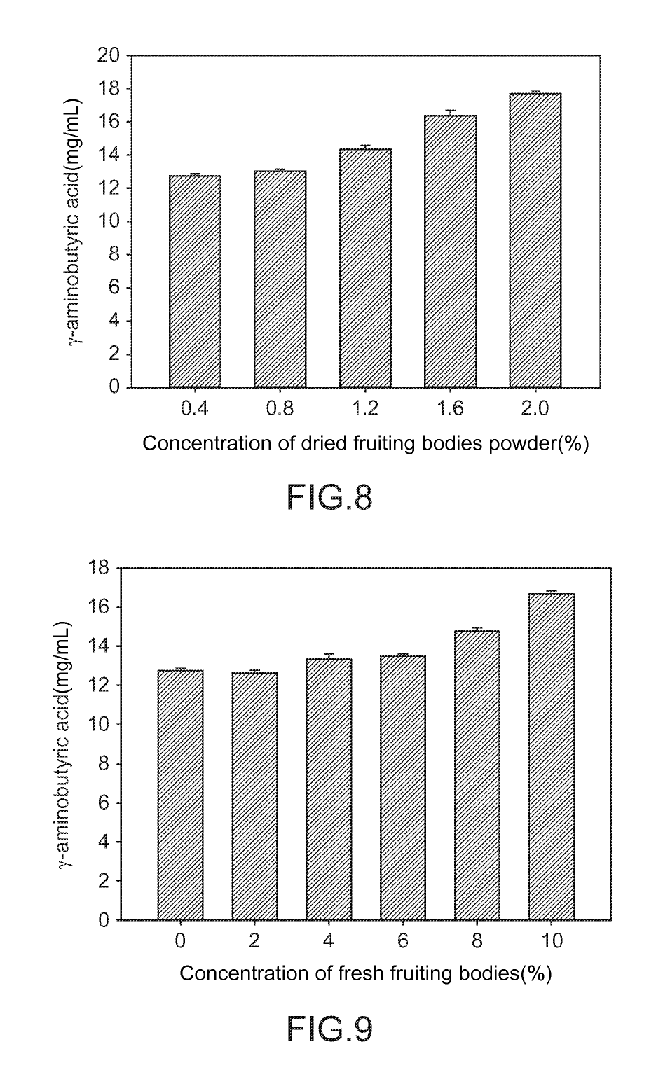

FIG. 8 is a bar chart showing the content of γ-aminobutyric acid after the FPA 3709 strain ferments the rice medium suspensions containing different concentrations of dried fruiting bodies powder. The horizontal axis denotes the concentration of dried fruiting bodies powder. The vertical axis denotes the content of γ-aminobutyric acid. In accordance with the experimental results of FIG. 8, as the concentration of the dried fruiting bodies powder is increased, the productivity of γ-aminobutyric acid is increased. In the group containing 2% dried fruiting bodies powder, the productivity of γ-aminobutyric acid is the highest.

Experiment 6: Effect of the Fresh Fruiting Bodies of the Cordyceps Species on the Productivity of γ-aminobutyric Acid

Firstly, an appropriate amount of rice medium powders is weighed, and is placed into distilled water, which has been subjected to sterilization at 121° C. for 15 minutes. Consequently, a 20% of rice medium suspension is prepared. Then, appropriate amounts of fresh fruiting bodies of the Cordyceps militaris are added to the rice medium suspension in different bottles. Consequently, the concentrations of the fresh fruiting bodies in each bottle is 0%, 2%, 4%, 6%, 8% and 10% (i.e. the grams of the fresh fruiting bodies/the milliliter of the rice medium suspension). Then, lactic acid bacteria FPA 3709 is added to each rice medium suspension to arrive an initial concentration 5.0˜6.0 log CFU/mL. Then, incubate at 37° C. for 48 hours. The content of the γ-aminobutyric acid in the fermentation broth is analyzed by a high performance liquid chromatography (HPLC) technique.

FIG. 9 is a bar chart showing the content of γ-aminobutyric acid after the FPA 3709 strain ferments the rice medium suspensions containing different concentrations of fresh fruiting bodies. The horizontal axis denotes the concentration of fresh fruiting bodies. The vertical axis denotes the content of γ-aminobutyric acid. In accordance with the experimental results of FIG. 9, as the concentration of the fresh fruiting bodies is increased, the productivity of γ-aminobutyric acid is increased. In the group containing 10% fresh fruiting bodies, the productivity of γ-aminobutyric acid is the highest

Experiment 7: Effect of the Edible Chinese Herbal Medicine on the Productivity of γ-aminobutyric Acid

An appropriate weight of a Chinese herbal medicine and the five times of its weight of the distilled water (compared with the weight of the Chinese herbal medicine) are both placed in an inner pot of a rice cooker (TCA-10A), and two cups of water (approximately 340 mL) is placed in an outer pot. Cook for 40 minutes. After the cooked mixture of the Chinese herbal medicine and the distilled water is filtered by four layers of gauze, this filtrate is the 20% (w/v) cooked Chinese herbal medicine solution. The 20% (w/v) cooked Chinese herbal medicine solution is placed in a sterile serum bottle, stored at 4° C. (ready for use). Then, the 20% (w/v) cooked Chinese herbal medicine solution is diluted with different volume of 25% of rice medium suspension, so that a rice medium suspension containing 1%˜4% of cooked Chinese herbal medicine is prepared. Then, lactic acid bacteria FPA 3709 is added to the rice medium suspension to arrive an initial count 5.0˜6.0 log CFU/mL. Then, incubate at 37° C. for 48 hours. The content of the γ-aminobutyric acid in the fermentation broth is analyzed by a high performance liquid chromatography (HPLC) technique.

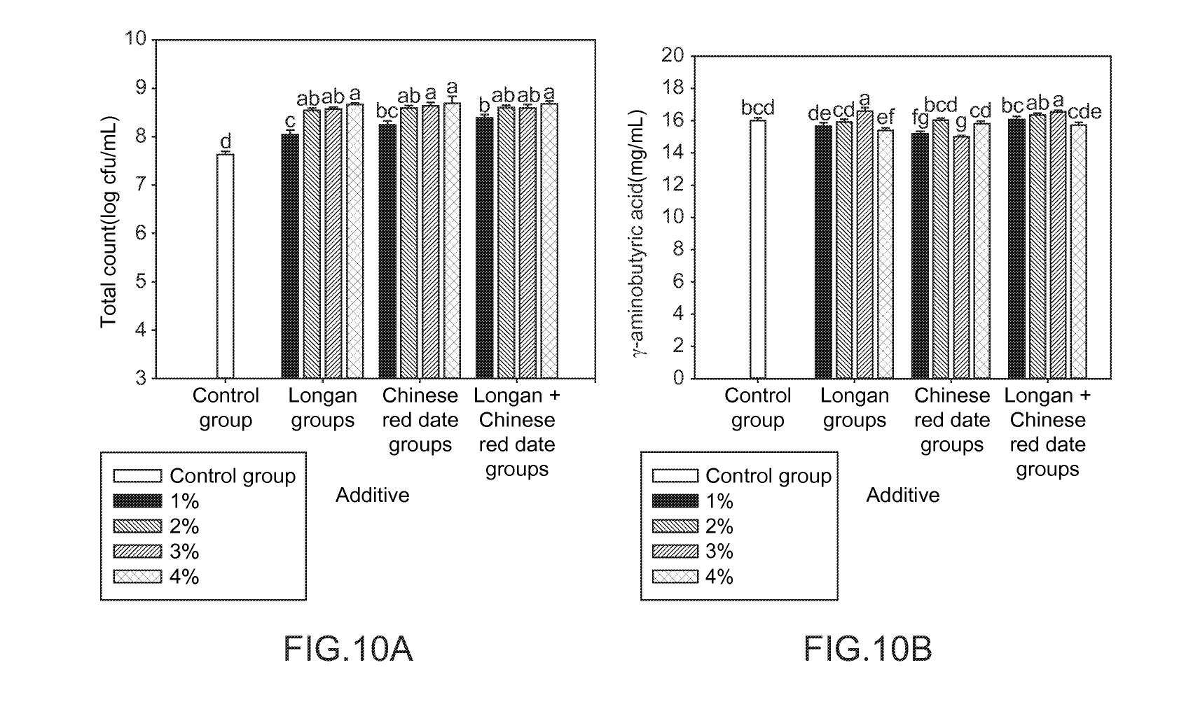

FIG. 10A is a bar chart showing the total counts of the FPA 3709 strain after it ferments the rice medium suspensions containing different concentrations of dried longan or Chinese red date. From left to right of the horizontal axis, control group, longan groups, Chinese red date groups, and longan and Chinese red date groups are sequentially shown. There are four different cooked Chinese herbal medicine concentrations (e.g. 1%, 2%, 3% and 4%) in each kind of groups. The vertical axis as shown in FIG. 10A denotes the total count of the lactic acid bacteria FPA 3709. FIG. 10B is a bar chart showing the content of γ-aminobutyric acid after the FPA 3709 strain ferments the rice medium suspensions containing different concentrations of dried longan or Chinese red date. The horizontal axis as shown in FIG. 10B is identical to the horizontal axis as shown in FIG. 10A. The vertical axis as shown in FIG. 10B denotes the content of γ-aminobutyric acid. In FIGS. 10A and 10B, if two groups of data are marked with the same superscript, there is no significant difference between these two groups.

In accordance with the experimental results of FIG. 10A, adding various concentration of the Chinese herbal medicine solution to the rice medium suspension can obviously increase the total count of the lactic acid bacteria FPA 3709 after fermentation. Moreover, the influences of the three kinds of cooked Chinese herbal medicine solutions on the total count of FPA 3709 are very similar (e.g. 8.54±0.05 log CFU/mL˜8.60±0.05 log CFU/mL). Even if the concentration of the added Chinese herbal medicine is increased to 4%, the total count of the lactic acid bacteria strain FPA 3709 is nearly unchanged. In accordance with the experimental results of FIG. 10B, different types and concentrations of the Chinese herbal medicine do not obviously influence the productivity of γ-aminobutyric acid.

The flavor and acidity of the fermentation broth may be influenced if add the Chinese herbal medicine solutions in different moments (for example, add before fermentation or add after fermentation). Fortunately, whenever do we add the Chinese herbal medicine solution, the Chinese herbal medicine solution can always diminish the sour taste and unpleasant smell of the rice medium, so that the fermentation broth or food is more acceptable for people to eat.

As previously described, the conventional method for treating the residual rice medium is detrimental to the ecological environment, pollutes the soil, and wastes the resources of the healthily-valuable mycelia of the Cordyceps species. By investigating the components of the rice medium, the inventors of the present invention found that the rice medium infiltrated by a large amount of Cordyceps species is more protein-rich. Moreover, additional lactic acid bacteria to the fermentation broth with the rice medium in it can increase the productivity of the γ-aminobutyric acid. Consequently, the present invention provides a novel method for producing γ-aminobutyric acid. Moreover, since the fermentation process can diminish the sour taste of the rice medium and enhance the overall favor thereof, the method of producing γ-aminobutyric acid by fermentation can be further applied in a novel health food or drink that comprises both of the mycelia of the Cordyceps species and the γ-aminobutyric acid, a novel health food or drink having the composite health value.

In the rice medium or in the material for preparing a food or drink, the concentration of said cordycepin is higher than 30 μg/mL (in drink), or the concentration of said cordycepin is higher than 90 μg/g (in food).

In the heath food or drink, the concentration of said γ-aminobutyric acid is higher than 0.1 mg/mL (in drink), or the concentration of said γ-aminobutyric acid is higher than 0.3 mg/g (in food). Preferably, the concentration of said γ-aminobutyric acid is higher than 3 mg/mL, or the concentration of said γ-aminobutyric acid is higher than 9 mg/g (in food).

The heath food or drink further comprises cordycepin, wherein the concentration of said cordycepin is higher than 10 μg/mL (in drink), or the concentration of said cordycepin is higher than 30 μg/g (in food). Preferably, the concentration of said cordycepin is higher than 30 μg/mL (in drink), or the concentration of said cordycepin is higher than 90 μg/g (in food).

Consequently, the method for producing γ-aminobutyric acid by fermenting the rice medium is helpful to increase the industrial application values of the rice medium, enhance the heath value of food, and eliminate the problems of the rice medium waste. Moreover, the method for producing γ-aminobutyric acid according to the present invention has many benefits such as promptness, mass production and cost-effectiveness.

From the above descriptions, the present invention uses Cordyceps militaris and the lactic acid bacteria with glutamate decarboxylase activity to produce γ-aminobutyric acid in the fermentation broth contain rice medium.

The rice medium, fresh fruiting bodies of the Cordyceps militaris, and the dried fruiting bodies of Cordyceps militaris can be purchased from Mu-Cho-Fu-Da International Co., Ltd. Of Taiwan.

Address: 1F., NO.107-1, SEC. 4, XINYI RD., DA-AN DISTRICT, TAIPEI CITY 10684, TAIWAN (R.O.C.)

TEL: 8862-2708-6177

WEB SITE: http://www.muchoherb.com/CF_sec01.html

The product name is “Sheng-Xian Cordyceps sinensis” and “Zhong-Hua golden fruiting body essence of Cordyceps sinensis, NO.1”.

The lactic acid bacteria can be purchased from Xin-Tai-An Technology Co., Ltd. Of Taiwan.

Address: 1F., No.19-13, Sanchong Rd., Nangang Dist., Taipei City 11501, Taiwan (R.O.C.);

TEL: 8862-2655-1551

The product number is FPP3713, FPA3708, FPA3709, FPM3702, FPS2520, FKR2526, FPAW3778, FKR3737, FKR3739, and FKR3741.

The lactic acid bacteria can also be purchased from online shopping channels, such as Ruten Auction.

The web site is http://class.ruten.com.tw/user/index00.php?s=cta8888;

The product number is FPP3713, FPA3708, FPA3709, FPM3702, FPS2520, FKR2526, FPAW3778, FKR3737, FKR3739, and FKR3741.

Since the biological materials involved “are known and readily available to the public” and “can be made or isolated without undue experimentation”, in accordance with 37 CFR 1.802(b), these biological materials need not be deposited.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for producing γ-aminobutyric acid, said method comprising steps of:
preparing a suspension of a residual rice medium; and
fermenting said suspension with a living Cordyceps species retained in said residual rice medium and/or with an externally-added microorganism to produce said γ-aminobutyric acid.

2. The method according to claim 1, wherein said residual rice medium is further ground into rice medium powders, and said rice medium powders are mixed with a liquid to form said suspension.

3. The method according to claim 1, wherein said residual rice medium is partially or completely infiltrated and replaced by mycelia of said Cordyceps species.

4. The method according to claim 3, wherein a crude protein content of said residual rice medium is higher than a crude protein content of the same type of original cooked rice medium material where no fruiting bodies of said Cordyceps species have been cultivated.

5. The method according to claim 4, wherein said crude protein content of said residual rice medium is 6%˜30% on a dry weight basis.

6. The method according to claim 1, wherein a weight/volume percent concentration of said residual rice medium of said suspension is lower than or equal to 60%.

7. The method according to claim 1, wherein said externally-added microorganism is a lactic acid bacteria.

8. The method according to claim 7, wherein said lactic acid bacteria is Lactobacillus brevis.

9. The method according to claim 1, wherein said externally-added microorganism is an externally-added Cordyceps species.

10. The method according to claim 9, wherein a weight/volume percent concentration of said externally-added Cordyceps species of said suspension is lower than or equal to 50%.

11. A food or drink, containing γ-aminobutyric acid and cordycepin.

12. The food or drink according to claim 11, wherein the concentration of said γ-aminobutyric acid is higher than 0.1 mg/mL, or the concentration of said γ-aminobutyric acid is higher than 0.1 mg/mL and the concentration of said cordycepin is higher than 10 μg/mL, or the concentration of said γ-aminobutyric acid is higher than 0.3 mg/g and the concentration of said cordycepin is higher than30 μg/g.

13. The food or drink according to claim 11, wherein the concentration of said γ-aminobutyric acid is higher than 3 mg/mL, or the concentration of said γ-aminobutyric acid is higher than 9 mg/g.

14. The food or drink according to claim 11, wherein the concentration of said cordycepin is higher than 30 μg/mL, or the concentration of said cordycepin is higher than 90 μg/g.

15. The food or drink according to claim 11, wherein at least partial of said γ-aminobutyric acid is the product from fermentating a residual rice medium.

16. The food or drink according to claim 15, further comprising an edible material for harmonizing the odors or tastes of said residual rice medium, and/or for harmonizing the the odors or tastes from fermentation.

17. The food or drink according to claim 16, wherein said edible material is at least one selected from Chinese red date, dried longan, and edible Chinese herbal medicine.

18. The food or drink according to claim 16, wherein said edible material is at least one selected from a Chinese red date extract, a dried longan extract, and an edible Chinese herbal medicine extract.

19. A material for preparing a food or drink, said material comprising:
a starchy material obtained from a plant tissue and/or from a cereal; and
fungal mycelia and/or cordycepin.

20. The material according to claim 19, wherein the concentration of said cordycepin is higher than 90 μg/g, or the concentration of said cordycepin is higher than 30 μg/mL.

21. The material according to claim 19, wherein said starchy material is a residual starchy medium, wherein said residual starchy medium is obtained by growing a Cordyceps species on a starchy medium and cutting off fruiting bodies of said Cordyceps species from said starchy medium to leave said residual starchy medium.
